(19) United States
(12) Patent Application Publication
(10) Pub. No.: US 2018/0132801 A1
(43) Pub. Date: May 17, 2018

(54) PATIENT POSITIONING APPARATUS

(71) Applicant: OXFORD UNIVERSITY INNOVATION LIMITED, Oxford (GB)

(72) Inventors: Thomas HAMILTON, Oxford (GB); David MURRAY, Oxford (GB); Stephen MELLON, Oxford (GB); Hemant PANDIT, Oxford (GB)

(21) Appl. No.: 15/569,052

(22) PCT Filed: Apr. 25, 2016
(86) PCT No.: PCT/IB2016/052340

§ 371 Date: Oct. 24, 2017

(30) Foreign Application Priority Data
Apr. 24, 2015 (GB) 1507059.2
Oct. 5, 2015 (GB) 15175586.5

(51) Int. Cl. A61B 6/04; A61G 13/12; A61B 6/00

(52) U.S. Cl. CPC A61B 6/0421; A61G 13/1245; A61B 5/4585; A61B 6/50; A61B 6/0485; A61B 6/4283

(57) ABSTRACT

A patient positioning apparatus for use with a medical imaging device, the patient positioning apparatus comprising a first element and a second element, the first element having a base part and an upper part to engage a knee joint of the patient, the second element comprising a lower leg engagement part to receive a lower leg of the patient to maintain the lower leg in a desired position.

This application relates to a patient positioning apparatus for use with a medical imaging device, and a method of imaging a patient using an imaging device, particularly but not exclusively to produce standardised knee stress X-rays.

### BACKGROUND TO THE INVENTION

Where a knee joint is suffering from arthritis, a known approach is to provide an artificial replacement joint. This may be a complete replacement, known as a total knee replacement (“TKR”) or a unicompartmental or partial knee replacement (“UKR”). To assess whether or not a knee joint is suitable for replacement by UKR or TKR, a standardised set of medical images, in particular X-rays, or radiographs, are used to assess the condition of the joint and the pattern and severity of arthritis affecting the knee. For example, for the Oxford UKR assessment, five radiographs are taken including an anterior-posterior weight bearing view, when the patient is standing, a true lateral view, sideways through the knee, a skyline view to image the patella-femoral joint, and views of the knee during valgus and varus stress. With the patient lying supine, face up, the varus stress images are taken by with the clinician fixing the knee in place and displacing the lower leg toward the centre of the body thus compressing the medial compartment, inside half, of the knee. In this position an image is taken enabling assessment of the thickness of the cartilage in the medial compartment and status of the lateral collateral ligament. The valgus stress images are taken with the clinician fixing the knee in place and by displacing the lower leg away from the centre line of the body thus compressing the lateral compartment, outside half, of the knee. In this position and image is taken enabling assessment of the thickness of the cartilage in the lateral compartment and the state of the medial collateral ligament. The thickness of the cartilage in each compartment in turn can be measured and can range from full thickness cartilage to complete cartilage loss, indicated by bone on bone contact.

This approach has disadvantages, not least that it is uncomfortable for the patient. The requirement for the presence of the clinician can be time-consuming, and exposes the clinician to additional X-rays with a consequent health risk. In addition, the use of manual intervention means that a subjective element is necessarily introduced into the process by the manual positioning of the lower legs and interpretation of the resulting images. Attempts have been made to produce devices to overcome these disadvantages but these are complex and not user-friendly. It is also known for surgeons to rely on standing X-ray views but none of these views are optimal for establishing a pattern or severity of disease, particularly in the lateral compartment, and additional diagnostics including magnetic resonance imaging (“MRI”) and arthroscopy (keyhole surgery) are often performed to confirm suitability for any replacement operation. However this approach has cost implications as well as resulting in patients having to wait longer to undergo surgery as result of the further diagnostic steps.

### SUMMARY OF THE INVENTION

According to a first aspect of the invention there is provided a patient positioning apparatus for use with a medical imaging device. The patient positioning apparatus comprising a first element and a second element, the first element having a base part and an upper part to engage the knee joints of the patient, the second element comprising a lower leg engagement part to receive a lower leg of the patient to maintain the lower leg in a desired position.

The patient positioning apparatus may further comprise a separation element to engage the first element, comprising an upright part to be received between knee joints of the patient.

The first element may comprise a channel in the upper part to receive the separation element or an X-ray cassette.

The first element may comprise a connector to engage the separation element.

The upper part of the first element may be substantially triangular in cross section.

The base part of the first element may be substantially flat to engage a patient support surface.

The base part may have a recess to receive an x-ray cassette.

The second element may comprise a first receiving part to receive a left lower leg of the patient, and a second receiving part to receive a right lower leg of the patient.

The first and second receiving parts may be separated by a divider.

The second element may comprise a main body, wherein the first receiving part and second receiving part comprise recesses in the main body.

The first receiving part and second receiving part may comprise recesses shaped to receive the feet and/or ankles of a patient.

The second element may comprise spaced first and second leg engagement parts and an expansion mechanism to provide a force on the first and second leg engagement parts.

The expansion mechanism may be operable to move the first and second leg engagement parts between a closed position and an expanded position.

The separation element may comprise the second element.

The first element and second element may be substantially radio-transparent.

The first element and second element may comprise a foam material.

According to a second aspect of the invention there is provided a method of imaging a patient using an imaging device, comprising providing a patient positioning apparatus according to any one of the preceding claims, disposing the first element and second element on a patient support surface, locating the patient in a recumbent position on the patient support surface such that the upper part of the first element engages the rear of the patient's knee joints and supports the knee joints and forming an image using the imaging device.

The method may comprise locating a separation element in engagement with the first element between the patient's knees before forming the image.

The method may comprise engaging the lower limbs of the patient in the second element, and forming an image using the imaging device.

The method may comprise locating the second element in a closed configuration between the patient's thighs and expanding the second element before forming the image.

The method may comprise locating the second element in a closed configuration between the patient's lower legs and expanding the second element before forming the image.

### BRIEF DESCRIPTION OF THE DRAWINGS

An embodiment of the invention is described by way of example only with reference to the accompanying drawings, wherein;

FIG. 1 is a perspective view of a first element embodying the present invention,

FIG. 2 is a perspective view of a second element embodying the present invention,

FIG. 3 is a perspective view of a separation element for use with the first element of FIG. 1,

FIG. 4 is a view of a force strap,

FIG. 5A is a view of a further second element embodying the invention in a closed configuration,

FIG. 5B is a view of the second element of FIG. 5A in an expanded configuration,

FIGS. 6A and 6B illustrate performing a valgus stress test using the second element of FIG. 5A, and

FIGS. 7A and 7B illustrate performing a varus stress test using the second element of FIG. 5A.

### DETAILED DESCRIPTION OF THE PREFERRED EMBODIMENTS

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

In the following description and claims, the term “lower leg” is intended to be generally interpreted to mean any or all of the calf, shin, ankle, heel and foot of a patient.

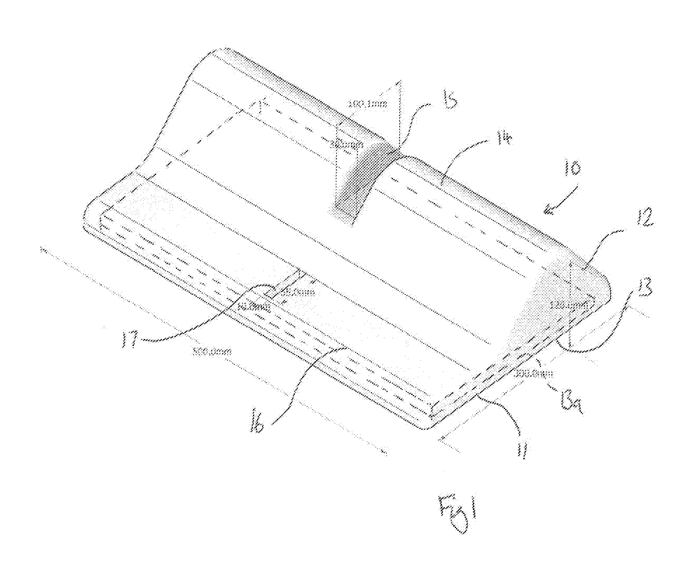

Referring to FIG. 1, a first element is generally shown at 10. The element 10 includes a base part 11 and an upper part 12. The base part 11 has a flat lower surface 13, to engage a patient support surface, such as which a patient is positioned during imaging. Additionally, in this example the base part 11 has a recess, here shown in dashed outline at 13a, of a sufficient size to receive an X-ray cartridge. Some radiology machines have a recess to receive an X-ray cartridge within a patient support surface, such as a radiology table, or under it. The upper part 12 is generally triangular in cross-section, with the uppermost part 14 being curved for patient comfort. At the midpoint of the first part 10, a slot 15 is provided extending through the upper part 12. The slot is sized to receive one of an X-ray cassette as discussed in more detail below, or a separation element as shown in FIG. 3. The base part 11 has an extension 16 which extends beyond the base of the upper part 12. The extension has a slot 17, broadly in line with the channel 15. The slot 17 is provided to hold to allow the attachment of a strap thereto to apply a constant force to the patient's legs to perform valgus and varus stress tests as will be discussed in more detail below.

In the present example, the overall height of the first element 10 from base 13 to upper part 14 is 120 mm, and it has a length, that is perpendicular to the ridge of upper part 12, of 300 mm. The first part 10 has a width, parallel to the upper part 12, of 500 mm, and the channel 15 has a width of 30 mm. The slot 17 is 55 mm long and 10 mm wide. The height of the upper part 12, and in particular the distance from upper surface 14 to the base 13, is selected such that, when the knee of a patient is resting on the first part 10 such that the upper surface 14 engages and supports the rear of the patient's knee joint, the knee joint is held at a constant and desired height. Advantageously, the first element may include calibration markers to permit measurement of the images, such as radio-opaque elements which will appear on X-ray images. The first element may also include anatomical side markers to designate the side of the body being imaged, preferably within the upper part 14 of the first element 10.

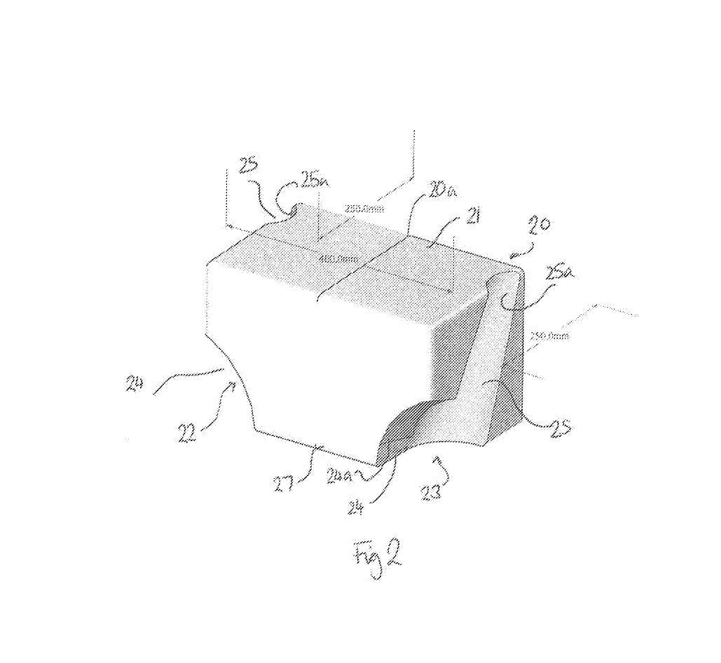

Referring to FIG. 2, a second element is shown at 20. The second element comprises a main body generally shown at 21. A first receiving part is generally shown at 22, comprising a recess to receive the left lower leg of the patient. A second receiving part comprising a second recess is generally shown at 23, to receive the right lower leg of the patient. As best seen is in connection with the second receiving part 23, each recess comprises a first generally horizontally extending channel 24 to engage the ankle of a patient, and an upwardly extending part 25 in which the foot of the patient is received. The channel 24 has an inner surface 24a which will engage the patient's ankle, and the upwardly extending part 25 again has an inclined inner face 25a against which the side of the patient's foot will rest. The inner faces 25a are inclined such that each is further from a centreline 20a of the main body 21 at the top and closer to the centreline 20a at the bottom. The angle of surfaces 25a is selected such that when the patient's feet are received in the first and second receiving parts 22, 23 and are engaged with the respective inclined face 25a, the patient's feet are held in position and rotated such that they are directed at an angle of approximately 15° outwards from the heels. A lower surface 26 of the main body 21 is provided to engage a patient support surface, and a divider 27, comprising part of the main body 21, provides a fixed spacing between the first and second holding parts.

It will be apparent that, in use, the first and second elements 10 maintain the patient's knees in a constant and standard angle of flexion, in this example about 20°, and the patient's feet at a constant separation and angle, set by the divider 27 between the recesses 22, 23 and the angle of the respective surfaces 25a.

A separation element is shown at 30 in FIG. 3. In this example, the separation element has a substantially rectangular body 31 with, at a lower side, a substantially triangular recess 32 to engage the upper part 12 of the first element 10. A bar 33 is located within the recess 32 and is shaped to engage the slot 15 of the first element 10 to hold the separation element in place. In this example, the separation element has a width of 120 mm, but it will be apparent that any other width or shapes may be used as appropriate, such as being wedge-shaped to fit between a patient's thighs.

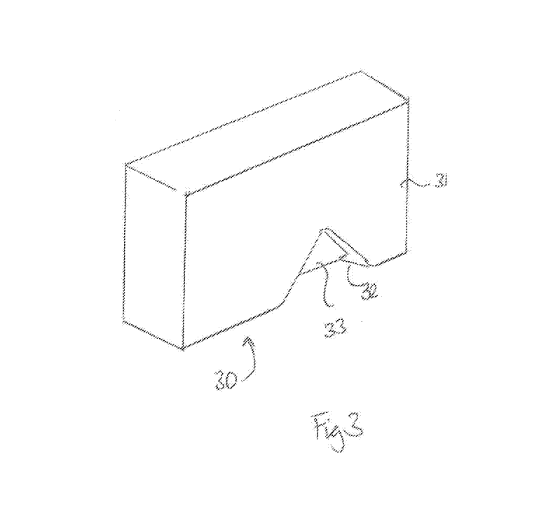

A force strap is shown at 40 in FIG. 4. The force strap 40 comprises a broad elongate band 41 which is sufficiently long to pass around a patient's thighs, and a tensioner 42 to tighten the force strap 40 around the patient's legs when in position. In this example the force strap 40 comprises a force gauge 43 to show the force being applied, although any other indicator could be provided such as a simple two-state indicator which changes state to show when the desired force has been achieved. The band 41 comprises a material that is able to tolerate force up to 150N without deforming significantly or breaking. The material should permit frequent operation without wear, be safe for skin contact and offer some padding to the skin. In this example the band 41 comprises 50 mm wide polypropylene webbing with a 50 mm plastic (polyoxymethylene, Delrin) buckle and a force gauge 43 that records up to 100N.

The first and second elements 10, 20 are used to generate images of the patient's knees under valgus stress in the following manner. Using a medical imaging device, in the present example an X-ray machine, the first and second elements 10, 20 are placed on a patient support surface and the patient is located in a recumbent position in which their knee joints are supported on the first element 10 such that the upper surface 14 engages and supports the knee joints, and their feet are received in the respective holding parts 22, 23 of the second element 20. An X-ray cassette is placed in the recess 13a in the base 11 of the first part 10 or under or within the patient support surface. A strap (not shown) attached to channel 17 is attached around both legs 100 mm proximal to the proximal pole of the patella, and a suitable force, generally between 30 to 50 N of force and in this example 40 N of force, is applied using the force strap 40. This can be verified, for example from the force gauge 43 in the strap. Accordingly, this draws the patient's knees towards one another under a known force while maintaining their feet at a known position and separation. An X-ray image is then taken of one or both knee joints as desired, replacing the X-ray cassette between images.

To perform a varus stress view, the second element 20 is then removed. A separation element 30 is then inserted in slot 15, between the knees, having a width of in this example 120 mm. A strap is placed around the lower legs to draw the feet together while the knees engage the separation element 30. Again, the strap has in this example has a force gauge to ensure that a constant force is applied. An X-ray cassette is located in the recess 13a, or under or within the patient support surface to enable the images to be taken.

The first and second elements 10, 20 are made of a suitable material. Ideally, the material is substantially radio-transparent. In the present example, the material comprises a closed cell foam to provide for patient comfort and infection control. Alternatively, the first and second elements could be formed from respective solid blocks of injection moulded polymer and subsequently formed or routed. The separation element may be made out of a similar material. Further alternatively, the first and second elements may be inflatable to the correct shape, and may be inflated with any suitable gas or liquid as appropriate.

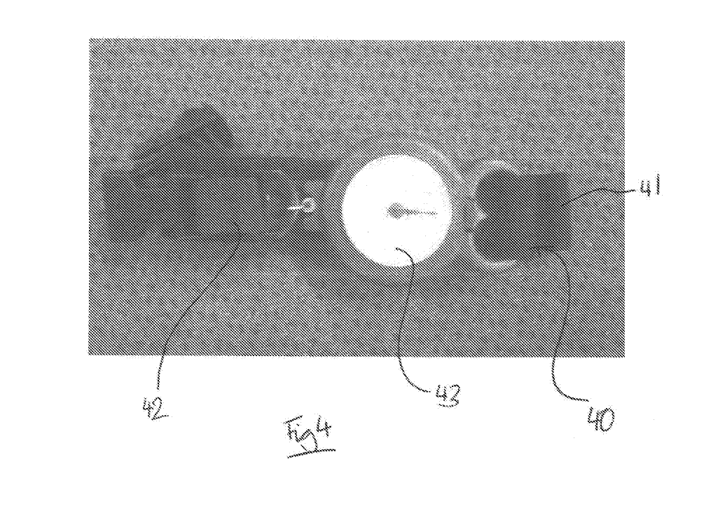

Another embodiment is shown in FIGS. 5A to 7B. In this embodiment, the second element comprises an expandable unit shown at 50. As will be described below, the expandable unit is used with a first element 10 and force strap 40 as described above. The expandable unit 50 is the primary means of applying force to the legs, and force strap 40 serves to maintain the separation of the legs and measure the applied force.

Referring now to FIGS. 5A and 5B, the expandable unit 50 comprises a first leg engagement part 51a and a second leg engagement part 51b, spaced from one another and connected by an expansion mechanism 52 with an operator control 53 to apply an outward force to the leg engagement parts. In the example shown the first and second leg engagement parts 51a, 51b are gently contoured in a curve and padded such that they engage with the contour of the inner leg, and measure 150 mm (height) by 150 mm (width) though with appropriate padding plates measuring 150 mm (height) by 50 mm (width) would be expected to apply an appropriate force. Optionally, the angle of the line from the top of each first and second leg engagement part 51a, 51b to the bottom will represent the optimum angle that the foot needs to be rotated to align a lateral X-ray of the knee. The first and second leg engagement parts 51a, 51b may comprise a suitable material which is radiolucent, have a smooth surface to permit cleaning and be safe for skin contact, for example a closed-cell foam material similar to that of the first element 10.

The expansion mechanism 52 in this example is a scissor jack mechanism, where lattice arms 52a, 52b are pivotally connected by bars 52c, 52d. Operator control 53 comprises a threaded rod 53a passing through threaded holes in bars 52c, 52d, such that rotation of a handle 53b changes the spacing of the bars 52c, 52d and opens or closes the lattice arms 52a, 52b, changing the spacing of the first and second leg engagement parts 51a, 51b. The expansion mechanism is operable to move the first and second leg engagement parts between a closed position and an expanded position. The expandable unit is shown in a fully closed position in FIG. 5A and in a fully open position in FIG. 5B. It will be apparent that any other mechanism suitable for moving the first and second leg engagement parts and for generating a controllable outward force on the legs as discussed below may be provided.

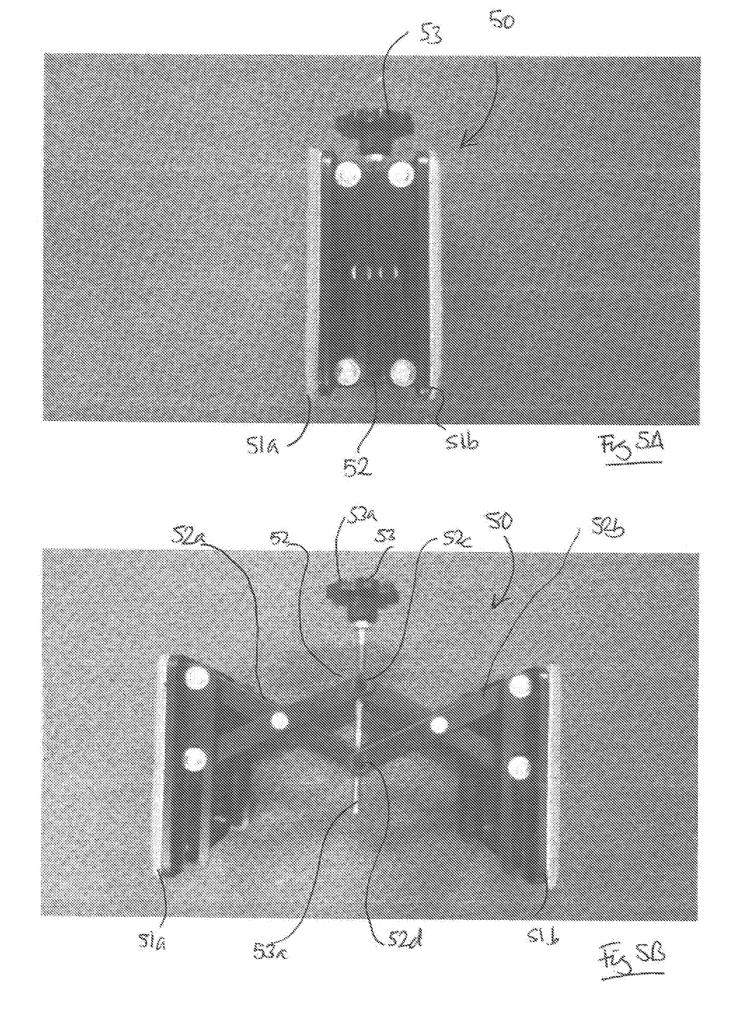

To perform a valgus stress view, the elements may be used as illustrated in FIGS. 6A and 6B. Using a medical imaging device, in the present example an X-ray machine, the first element 10 is placed on a patient support surface and the patient is located in a recumbent position in which their knee joints are supported on the first element 10 such that the upper surface 14 engages and supports the knee joints. The expandable unit 50 is initially in a fully closed configuration as shown in FIG. 5A is place between the calves 100 mm distal to the knee joint and the legs brought together either side of the expandable unit 50. A force strap 40 is attached around both thighs, in this example about 100 mm proximal to the proximal pole of the patella, and an initial tension applied. The expandable unit 50 is then expanded by turning the handle 53b to apply an outward force on the calves until a suitable force, generally between 30 to 50 N of force and in this example 50 N of force, is applied. This can be verified, for example from the force gauge 43 in the strap. Accordingly, this draws the patient's knees towards one another under a known force while maintaining their feet at a known position and separation. Preferably, the alignment of the leg is checked by the operator to ensure the tibial tubercles are facing anteriorly, and the X-ray beam is directed 10 degrees cephalic in the coronal plane, centred on the knee. An X-ray image is then taken of one or both knee joints as desired.

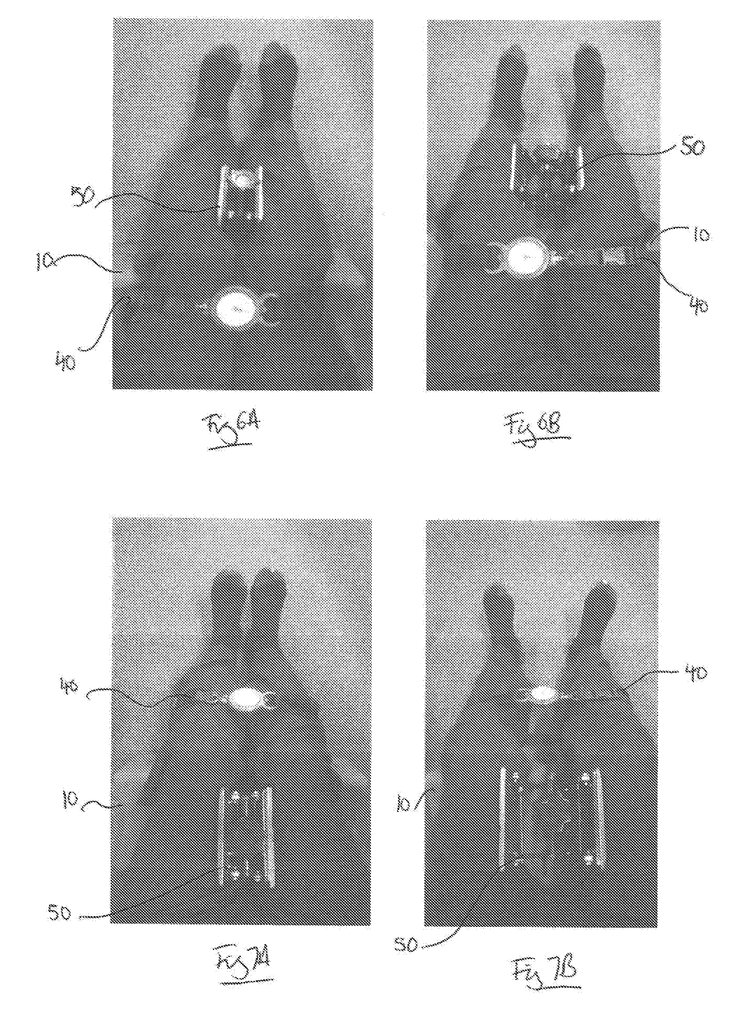

To perform a varus stress view, the elements may be used as shown in FIGS. 7A and 7B. Expandable unit 50 in its closed configuration is located between the thighs, in this example about 100 mm proximal to the knee joint. Force strap 40 is placed around the lower legs, in this case the shins, to draw the feet together while the thighs engage the expandable element 50. The expandable unit 50 is then expanded until the force measure by the force gauge 53 reaches an appropriate force, in this example 50N. Preferably, the alignment of the leg is checked by the operator to ensure the tibial tubercles are facing anteriorly, and the X-ray beam is directed 10 degrees cephalic in the coronal plane, centred on the knee. An X-ray image is then taken of one or both knee joints as desired. In this method, it will be apparent that the expandable unit 50 replaces the separation element 30.

It will be apparent that the use of the apparatus described herein applies a standard force to the knees, using the opposite limb as an anchor from which to apply these forces. The flexion and rotation of the limbs are standardised and so give valgus and varus stress images with standardised stresses applied to the joints, and with the joints positioned in a standard and optimal pattern. The standard views obtained using the apparatus mean that no clinician is required to be present during X-rays, and the subjective component involved in positioning the patient's limbs is removed.

Although the apparatus is particularly described with reference to obtaining valgus and varus stress images of a knee joint, it will be apparent that the use of the apparatus need not be limited in this way. For example, the apparatus may be used to position the patient to obtain a horizontal beam lateral X-ray of the knee, in which case an X-ray cassette could be placed in recess 15 for a lateral X-ray. It may be also used to assess for medial collateral ligament insufficiencies as the applied stress would cause opening of the medial and lateral joints basis which would be apparent in the resulting images.

In the above description, an embodiment is an example or implementation of the invention. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

Meanings of technical and scientific terms used herein are to be commonly understood as by one of ordinary skill in the art to which the invention belongs, unless otherwise defined.

1. A patient positioning apparatus for use with a medical imaging device, the patient positioning apparatus comprising a first element and a second element,
the first element having a base part and an upper part to engage a knee joint of the patient,
the second element comprising a lower leg engagement part to receive a lower leg of the patient to maintain the lower leg in a desired position.

2. The patient positioning apparatus of claim 1 further comprising a separation element to engage the first element, comprising an upright part to be received between knee joints of the patient.

3. The patient positioning apparatus claim 2 wherein the first element comprises a channel in the upper part to receive the separation element.

4. The patient positioning apparatus of claim 2, wherein the first element comprises a channel in the upper part to receive the separation element and the channel is shaped to receive an x-ray cassette.

5. The patient positioning apparatus of claim 1 wherein the upper part of the first element is substantially triangular in cross section.

6. The patient positioning apparatus of claim 1 wherein the base part of the first element is substantially flat to engage a patient support surface.

7. The patient positioning apparatus of claim 6 wherein the base part has a recess to receive an x-ray cassette.

8. The patient positioning apparatus of claim 1 wherein the second element comprises a first receiving part to receive a left lower leg of the patient, and a second receiving part to receive a right lower leg of the patient, preferably wherein the first and second receiving parts are separated by a divider.

9. (canceled)

10. The patient positioning apparatus of claim 8 wherein the second element comprising a main body, wherein the first receiving part and second receiving part comprise recesses in the main body.

11. The patient positioning apparatus of claim 8 wherein the second element comprising a main body, wherein the first receiving part and second receiving part comprise recesses in the main body and wherein the first receiving part and second receiving part comprises recesses shaped to receive the feet and ankles of a patient.

12. The patient positioning apparatus of claim 1 wherein the second element comprises spaced first and second leg engagement parts and an expansion mechanism to provide a force on the first and second leg engagement parts.

13. The patient positioning apparatus of claim 12 wherein the expansion mechanism is operable to move the first and second leg engagement parts between a closed position and an expanded position.

14. The patient positioning apparatus of claim 2 wherein the separation element comprises the second element.

15. The patient positioning apparatus of claim 1 wherein the first element and second element are substantially radio-transparent.

16. The patient positioning apparatus of claim 1 wherein the first element and second element comprise a foam material.

17. A method of imaging a patient in an imaging device, comprising providing a patient positioning apparatus according to any one of the preceding claims,
disposing the first element on a patient support surface,
locating the patient in a recumbent position on the patient support surface such that the upper part of the first element engages the rear of the patient's knee joints and supports the knee joints,
and forming an image using the imaging device.

18. A method according to claim 17 comprising locating a separation element in engagement with the first element between the patient's knees before forming the image.

19. A method according to claim 17 comprising engaging the lower limbs of the patient in the second element, and forming an image using the imaging device.

20. A method according to claim 17 comprising locating the second element in a closed configuration between the patient's thighs and expanding the second element before forming the image.

21. A method according to claim 17 or claim 20 comprising locating the second element in a closed configuration between the patient's lower legs and expanding the second element before forming the image.
